(19) United States
(12) Patent Application Publication
(10) Pub. No.: US 2024/0010587 A1
(43) Pub. Date: Jan. 11, 2024

(54) Process for the preparation of dicidal

(71) Applicant: EVONIK OPERATIONS GMBH, Essen (DE)

(72) Inventors: Robert Franke, Marl (DE); Carolin Schneider, Monheim am Rhein (DE); Ralf Jackstell, Rostock (DE); Matthias Beller, Ostseebad Nienhagen (DE); Dieter Reusch, Marl (DE); Harald Häger, Lüdinghausen (DE)

(73) Assignee: EVONIK OPERATIONS GMBH, Essen (DE)

(21) Appl. No.: 18/345,424

(22) Filed: Jun. 30, 2023

(30) Foreign Application Priority Data
Jul. 6, 2022 (EP) 22183347.8

(51) Int. Cl. C07C 31/27

(52) U.S. Cl. CPC C07C 31/278; C07C 2531/12

(57) ABSTRACT

Process for the preparation of dicidal.

The present invention relates to a process for the preparation of dicidal.

US 2009/0171125 A1 describes a process for the hydroformylation of cyclic olefins. In this case an Rh catalyst is used.

The present invention has the object of providing a novel hydroformylation process. The process should afford an increased yield in comparison to the method known from the prior art.

This object is achieved by a process according to Claim 1.

Process comprising the process steps of:
a) initially charging dicyclopentadiene;
b) adding a compound of formula (I):

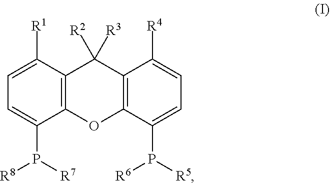

where R¹, R², R³, R⁴, R⁵, R⁶, R⁷, R⁸ are selected from: —H, —(C₁-C₁₂)-alkyl, -Ph;
c) adding a Pt compound capable of forming a complex;
d) adding an iodine compound;
e) feeding in CO and H₂;
f) heating the reaction mixture from steps a) to e), to convert the dicyclopentadiene to dicidal.

In this process, process steps a) to e) can be effected in any desired sequence. Typically, however, CO and H₂ are added after the co-reactants have been initially charged in steps a) to d).

Here, process steps c) and d) may also be carried out in one step, for example by adding PtI₂.

In one variant of the process, the Pt compound and the iodine compound are added in one step by adding PtI₂.

The expression (C₁-C₁₂)-alkyl encompasses straight-chain and branched alkyl groups having 1 to 12 carbon atoms. These are preferably (C₁-C₈)-alkyl groups, more preferably (C₁-C₆)-alkyl, most preferably (C₁-C₄)-alkyl.

Suitable (C₁-C₁₂)-alkyl groups are especially methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1,1,2-trimethylpropyl, 1,2,2-trimethylpropyl, 1-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

In one variant of the process, R¹ and R⁴ are —H.

In one variant of the process, R⁵, R⁶, R⁷, R⁸ are -Ph.

In one variant of the process, R² and R³ are —(C₁-C₁₂)-alkyl.

In one variant of the process, R² and R³ are —CH₃.

In one variant of the process, the compound (1) has the structure (1):

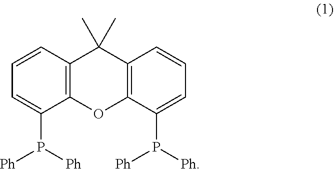 [SMILES: CC1(C)c2cccc(P(c3ccccc3)c3ccccc3)c2Oc2c(P(c3ccccc3)c3ccccc3)cccc21]

In one variant of the process, the Pt compound is selected from: Pt(II)I₂, Pt(IV)I₄, diphenyl(1,5-COD)Pt(II), Pt(II)(acac)₂, Pt(0)(PPh₃)₄, Pt(0)(DVTS) solution (CAS: 68478-92-2), Pt(0)(ethylene)(PPh₃)₂, tris(benzylideneacetone)Pt(0), Pt(II)(OAC)₂ solution, Pt(0)(t-Bu)₂, Pt(II)(COD)Me₂, Pt(II)(COD)I₂, Pt(IV)IMe₃, Pt(II)(hexafluoroacetylacetonate)₂.

In one variant of the process, the Pt compound is selected from: Pt(II)I₂, Pt(II)(acac)₂.

In one variant of the process, the Pt compound is Pt(II)I₂.

In one variant of the process, the iodine compound is selected from: alkali metal halide, alkaline earth metal halide, NH₄X, alkylammonium halide, dialkyl halide, trialkyl halide, tetraalkyl halide, cycloalkylammonium halide.

In one variant of the process, the iodine compound is selected from: Pt(II)I₂, LiI.

In one variant of the process, PtI₂ is added in an amount, measured in mol % based on dicyclopentadiene, such that the value is in the range from 0.1 mol % to 5 mol %.

In one variant of the process, PtI₂ is added in an amount, measured in mol % based on dicyclopentadiene, such that the value is in the range from 0.1 mol % to 3 mol %.

In one variant of the process, PtI₂ is added in an amount, measured in mol % based on dicyclopentadiene, such that the value is in the range from 0.1 mol % to 1 mol %.

In one variant of the process, this process comprises the additional process step e′):
e′) adding a solvent.

In one variant of the process, the solvent is selected from: THF, DCM, ACN, heptane, DMF, toluene, texanol, pentane, hexane, octane, isooctane, decane, dodecane, cyclohexane, benzene, xylene, marlotherm, propylene carbonate, MTBE, diglyme, triglyme, diethyl ether, dioxane, isopropanol, tert-butanol, isononanol, isobutanol, isopentanol, ethyl acetate.

In one variant of the process, the solvent is selected from: THF, DCM, ACN, heptane, DMF, toluene, texanol.

In one variant of the process, CO and H₂ is fed in at a pressure in a range from 1 MPa (10 bar) to 6 MPa (60 bar).

In one variant of the process, CO and H₂ is fed in at a pressure in a range from 2 MPa (20 bar) to 5 MPa (50 bar).

In one variant of the process, the reaction mixture is heated to a temperature in the range from 25° C. to 150° C.

In one variant of the process, the reaction mixture is heated to a temperature in the range from 30° C. to 130° C.

In one variant of the process, the process comprises the additional process step g):
g) converting dicidal to dicidol.

In one variant of the process, the conversion of dicidal to dicidol is carried out using “Shvo's catalyst” (CAS 104439-77-2).

The invention is to be illustrated in detail hereinafter by a working example.

### EXPERIMENTAL DESCRIPTION

Conversion of Dicyclopentadiene to Dicidal

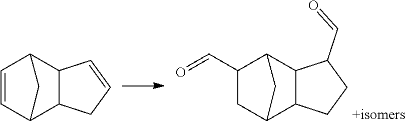 [SMILES: C1=CC2C3C=CC(C3)C2C1 ; O=CC1CC2CC1C1C(C=O)CCC21]

10 mmol of dicyclopentadiene (DCPD), 10 ml of absolute toluene, 0.5 mol % PtI₂, 2.2 equivalents of xantphos (1) (based on Pt) are placed under argon in a 25 ml steel autoclave from Parr Instruments. The autoclave was pressurized to 40 bar with synthesis gas (CO/H₂=1:1) and the reaction started by heating to 80° C. and stirring. This reaction is conducted at 40 bar/80° C. for 18.5 h. The autoclave is then cooled, the pressure released and a GC sample is taken.

In a comparative example, Rh(acac)(CO)₂ was added instead of PtI₂.

Yield of Dicidal:
PtI₂: 89.5%
Rh(acac)(CO)₂: <5%

Conversion of Dicidal to Dicidol

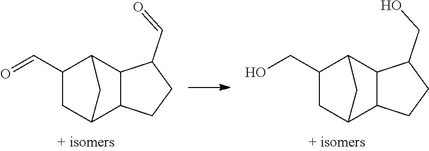 [SMILES: O=CC1CC2CC1C1C(C=O)CCC21 ; OCC1CC2CC1C1C(CO)CCC21]

10 ml of toluene, 5 ml of ethanol, 5.5 g of dicidal (28.6 mmol) and 62.16 mg of “Shvo's catalyst” (CAS 104439-77-2) (0.2 mol % with respect to dicidal) are heated to 100° C. under argon in a 100 ml Parr pressure autoclave at 40 bar hydrogen and reacted while stirring for 20 hours. The reaction is then stopped, the pressure released and the reaction mixture distilled in a fine vacuum. A colourless oil of the isomeric mixture of dicidol is obtained as a fraction at 140° C.

Yield of dicidol: 85%

As the experimental results show, the object is achieved by the inventive process.

1. Process comprising the process steps of:
a) initially charging dicyclopentadiene;
b) adding a compound of formula (I): 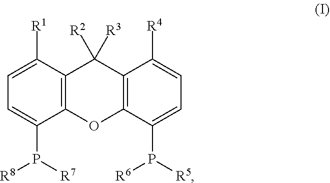
where R¹, R², R³, R⁴, R⁵, R⁶, R⁷, R⁸ are selected from: —H, —(C₁-C₁₂)-alkyl, -Ph;
c) adding a Pt compound capable of forming a complex;
d) adding an iodine compound;
e) feeding in CO and H₂;
f) heating the reaction mixture from steps a) to e), to convert the dicyclopentadiene to dicidal.

2. Process according to claim 1,
where R¹ and R⁴ are —H.

3. Process according to claim 1,
where R⁵, R⁶, R⁷, R⁸ are -Ph.

4. Process according to claim 1,
where R² and R³ are —(C₁-C₁₂)-alkyl.

5. Process according to claim 1,
where R² and R³ are —CH₃.

6. Process according to claim 1,
wherein the compound (I) has the structure (1):

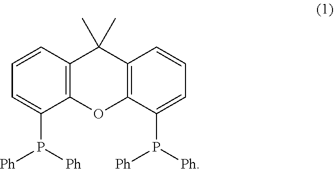 [SMILES: CC1(C)c2cccc(P(c3ccccc3)c3ccccc3)c2Oc2c(P(c3ccccc3)c3ccccc3)cccc21]

7. Process according to claim 1,
wherein the Pt compound is selected from: Pt(II)I₂, Pt(IV)I₄, diphenyl(1,5-COD)Pt(II), Pt(II)(acac)₂, Pt(0)(PPh₃)₄, Pt(0)(DVTS) solution (CAS: 68478-92-2), Pt(0)(ethylene)(PPh₃)₂, tris(benzylideneacetone)Pt(0), Pt(II)(OAC)₂ solution, Pt(0)(t-Bu)₂, Pt(II)(COD)Me₂, Pt(II)(COD)I₂, Pt(IV)IMe₃, Pt(II)(hexafluoroacetylacetonate)₂.

8. Process according to claim 1,
wherein the iodine compound is selected from: Pt(II)I₂, LiI.

9. Process according to claim 1,
wherein PtI₂ is added in an amount, measured in mol % based on dicyclopentadiene, such that the value is in the range from 0.1 mol % to 5 mol %.

10. Process according to claim 1,
comprising the additional process step e′):
e′) adding a solvent.

11. Process according to claim 1,
wherein CO and H₂ is fed in at a pressure in a range from 1 MPa (10 bar) to 6 MPa (60 bar).

12. Process according to claim 1,
wherein the reaction mixture is heated to a temperature in the range from 25° C. to 150° C.

13. Process according to claim 1,
wherein the process comprises the additional process step g):
g) converting dicidal to dicidol.

14. Process according to claim 13,
wherein the conversion of dicidal to dicidol is carried out using “Shvo's catalyst” (CAS 104439-77-2).
